(19) United States
(12) Patent Application Publication
(10) Pub. No.: US 2009/0030317 A1
(43) Pub. Date: Jan. 29, 2009

(54) ULTRASONIC IMAGING DEVICES, SYSTEMS, AND METHODS

(75) Inventor: Michael J. LEVY, Rochester, MN (US)

Correspondence Address: MUETING, RAASCH & GEBHARDT, P.A., P.O. BOX 581336, MINNEAPOLIS, MN, 55458-1336, US

(73) Assignee: Mayo Foundation for Medical Education and Research, Rochester, MN (US)

(21) Appl. No.: 12/173,357

(22) Filed: Jul. 15, 2008

Related U.S. Application Data
(60) Provisional application No. 60/961,939, filed on Jul. 25, 2007.

(51) Int. Cl. A61B 8/00

(52) U.S. Cl. 600/447

(57) ABSTRACT

Ultrasonic imaging devices, systems and methods that may be used to image difficult to reach locations such as internal body cavities, etc. The ultrasonic imaging devices may be embodied in endoscopes that include one ore more channels for delivering instruments and other items to the location at which the imaging is occurring or the imaging devices may be provided in the form of a probe or other device that may, for example, be delivered through a channel located in an endoscope.

### RELATED APPLICATION

The present application claims the benefit under 35 U.S.C. Section 119(e) of U.S. Provisional Patent Application Ser. No. 60/961,939 filed on Jul. 25, 2007 and titled ULTRASONIC IMAGING DEVICES, SYSTEMS, AND METHODS, which is hereby incorporated by reference in its entirety.

Ultrasonic endoscopes are used to examine the interior surfaces of body cavities and may be used for inspection as well as during treatment. Known endoscopes typically include either a linear ultrasonic array that extends along the longitudinal axis of the endoscope or a radial array that extends either completely around or partially around the body of the endoscope. In either case, the imaging elements are capable of providing only one type of planar cross-sectional view of the tissue or other structures surrounding the endoscope.

Attempts have been made to provide devices that are capable of providing two different planar cross-sectional views using two different ultrasonic imaging arrays. For example, the devices described in U.S. Pat. No. 6,171,248 (Hossack et al.) include both a longitudinal array aligned with the longitudinal axis of the device and a radial array extending at least partially around the circumference of the device. Both arrays are, however, capable of providing only one planar cross-sectional view. These planar cross-sectional views are combined and processed to provide a three-dimensional image of the tissue and other structures surrounding the device by moving the device along its longitudinal axis and/or rotating the device around its longitudinal axis. Such movements may, however, be difficult to control in the environments in which the devices are used. Furthermore, the probe does not include any channels capable of delivering other instruments or devices to the imaged location.

Another problem associated with ultrasonic imaging is that fluid-filled balloons are often used over the ultrasonic transducers to provide a liquid medium through which the ultrasound energy flows (air or other gases inhibit the propagation of ultrasonic energy). The liquids used to fill the balloons (e.g., saline, etc.), however, often contain bubbles and/or dissolved gases that form bubbles in the balloons. These bubbles can adversely affect the images obtained. Although the bubbles can be removed, it may take 5-30 minutes to do so, increasing cost, frustration, etc.

### SUMMARY OF THE INVENTION

The present invention provides ultrasonic imaging devices, systems and methods that may be used to image difficult to reach locations such as internal body cavities, etc. The ultrasonic imaging devices may be embodied in endoscopes that include one ore more channels for delivering instruments and other items to the location at which the imaging is occurring or the imaging devices may be provided in the form of a probe or other device that may, for example, be delivered through a channel located in an endoscope.

In some embodiments, the ultrasonic imaging devices may preferably include one or more two-dimensional arrays of ultrasonic elements (sometimes referred to as transducers). Each of the two-dimensional arrays occupies a selected area of the body on which they are located. Each of the two-dimensional arrays preferably includes two or more adjacent ultrasonic elements arranged in a first direction and two or more adjacent ultrasonic elements arranged in a second direction. The first direction may preferably be perpendicular to the second direction. A two-dimensional array may include any suitable number of ultrasonic elements, each of which may preferably be individually activatable.

For the purposes of illustration, an array may include twenty-four (24) ultrasonic elements arranged in a 4×6 array where the ultrasonic elements are arranged with four rows of six ultrasonic elements extending along a longitudinal axis of the device. The four rows of ultrasonic elements are arranged around the circumference of the device. This is, however, only one example of an array that may be used in connection with the present invention. The actual two-dimensional arrays used may have more or less than twenty-four ultrasonic elements arranged in more or less than four rows, each of which may contain more or less than six ultrasonic elements.

By activating selected sets of the ultrasonic elements in the two-dimensional arrays, different images may be obtained. For example, planar cross-sectional images in planes that are aligned with the longitudinal axis, perpendicular to the longitudinal axis, or canted somewhere between these two extremes may be obtained.

In some embodiments, the ultrasonic arrays may be used in devices (e.g., endoscopes) that include multiple ports located about the ultrasonic arrays, with the ports being useful to, e.g., deliver surgical devices, etc. It may be advantageous if the ports are arranged such that they align with the planar cross-sectional views obtained using the ultrasonic arrays. Such an arrangement may provide an improved image of instruments, etc. delivered through the ports.

In some embodiments, the ultrasonic elements may be covered by a balloon that is gas permeable. Because the balloon is permeable to gases, but not liquids, dissolved gases and bubbles may be able to exit the balloon without intervention by the user.

In one aspect, the present invention provides an ultrasonic imaging device that includes an elongated body defining a longitudinal axis extending from a proximal to a distal end of the body; a first ultrasonic array located proximate the distal end of the body, the first ultrasonic array including a plurality of ultrasonic elements arranged in a two-dimensional array over a selected area of the body, wherein, in a circumferential dimension, the two-dimensional array includes two or more adjacent ultrasonic elements arranged over a circumferential segment of the body, and wherein, in a longitudinal dimension, the two-dimensional array comprises two or more adjacent ultrasonic elements arranged over a longitudinal segment of the body. The ultrasonic elements of the first ultrasonic array are selectively activatable to obtain images in imaging planes, wherein at least one of the imaging planes is aligned with the longitudinal axis and at least one of the imaging planes is not aligned with the longitudinal axis. The device further includes a first port channel extending through the body, the first port channel having a first port located between the first ultrasonic array and the distal end of the body or between the first ultrasonic array and the proximal end of the body, wherein at least one of the imaging planes intersects with the first port.

In various embodiments, the devices of the present invention may include a second port channel extending through the body, the second port channel having a second port, wherein the first ultrasonic array is located between the first port and the second port, wherein at least one of the imaging planes intersects with the first port, the second port, and the longitudinal axis; a terminal port channel may extend through the body, the terminal port channel having a terminal port located at the distal end of the body, wherein at least one of the imaging planes intersects with the terminal port; the two-dimensional array of the first ultrasonic array may, in the circumferential dimension, include adjacent ultrasonic elements arranged around the entire circumference of the body; a balloon may be attached to the body, the balloon extending over the first ultrasonic array, wherein the balloon includes a gas permeable membrane, wherein gas contained within a liquid located between the body and the gas permeable membrane diffuses out of the liquid through the gas permeable membrane; etc.

In still other embodiments, the devices of the present invention may include a first side port channel extending through the body, the first side port channel having a first side port that is radially displaced from the first ultrasonic array, the first side port being located between the distal port and the proximal port along the longitudinal axis, wherein at least one of the imaging planes intersects with the first side port. The device may also include a second side port channel extending through the body, the second side port channel having a second side port that is radially displaced from the first ultrasonic array, the second side port being located between the distal port and the proximal port along the longitudinal axis, wherein the first ultrasonic array is located between the first side port and the second side port. When present, the first side port and the second side port may intersect with at least one of the imaging planes.

In still other embodiments, the devices of the present invention may include a second ultrasonic array located proximate the distal end of the body, wherein the second ultrasonic array includes a plurality of ultrasonic elements arranged in a two-dimensional array over a selected area of the body, wherein, in a circumferential dimension, the two-dimensional array includes two or more adjacent ultrasonic elements arranged over a circumferential segment of the body, and wherein, in a longitudinal dimension, the two-dimensional array also includes two or more adjacent ultrasonic elements arranged over a longitudinal segment of the body, and wherein the ultrasonic elements of the second ultrasonic array are selectively activatable to obtain images in imaging planes, wherein at least one of the imaging planes is aligned with the longitudinal axis and at least one of the imaging planes is not aligned with the longitudinal axis. The second ultrasonic array may be located on an opposite side of the body from the first ultrasonic array. The device may include a third port channel extending through the body, the third port channel having a third port, wherein at least one of the imaging planes obtained using the second ultrasonic array intersects with the third port. The device may include a fourth port channel extending through the body, the fourth port channel having a fourth port, wherein the second ultrasonic imaging array is located between the third port and the fourth port, and wherein at least one of the imaging planes obtained using the second ultrasonic array intersects with the third port, the fourth port, and the longitudinal axis.

In still another aspect, the present invention provides an ultrasonic imaging system including an ultrasonic imaging device with an elongated body defining a longitudinal axis extending from a proximal to a distal end of the body; a first ultrasonic array located proximate the distal end of the body, the first ultrasonic array having a plurality of ultrasonic elements arranged in a two-dimensional array over a selected area of the body, wherein, in a circumferential dimension, the two-dimensional array has two or more adjacent ultrasonic elements arranged over a circumferential segment of the body, and wherein, in a longitudinal dimension, the two-dimensional array has two or more adjacent ultrasonic elements arranged over a longitudinal segment of the body, and wherein the ultrasonic elements of the first ultrasonic array are selectively activatable to obtain images in imaging planes, wherein at least one of the imaging planes is aligned with the longitudinal axis and at least one of the imaging planes is not aligned with the longitudinal axis; and a first port channel extending through the body, the first port channel having a first port located between the first ultrasonic array and the distal end of the body or between the first ultrasonic array and the proximal end of the body, wherein at least one of the imaging planes intersects with the first port. The system further includes imaging electronics connected to the plurality of ultrasonic elements in the first ultrasonic array to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array.

In various embodiments, the imaging electronics may display a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane; the first imaging plane may be perpendicular to the second imaging plane; the imaging electronics may display the first visual image and the second visual image at the same time; etc.

In still other embodiments, at least one of the imaging planes may be canted with respect to the longitudinal axis of the imaging body.

In another aspect, the present invention may provide an endoscopic ultrasonic imaging system including an endoscope having an endoscope body defining a longitudinal axis extending from a proximal end to a distal end of the endoscope body; an imaging channel extending through the endoscope body, the imaging channel having an imaging window and, optionally, an alignment structure. The system further includes an imaging device sized for advancement through the imaging channel, the imaging device including an imaging body defining a longitudinal axis extending from a proximal end to a distal end of the imaging body; an ultrasonic array located proximate the distal end of the imaging body, the ultrasonic array having a plurality of ultrasonic elements arranged in a two-dimensional array over a selected area of the imaging body, wherein, in a circumferential dimension, the two-dimensional array has two or more adjacent ultrasonic elements arranged over a circumferential segment of the body, and wherein, in a longitudinal dimension, the two-dimensional array has two or more adjacent ultrasonic elements arranged over a longitudinal segment of the body, and wherein the ultrasonic elements of the ultrasonic array are selectively activatable to obtain images in imaging planes, wherein at least one of the imaging planes is aligned with the longitudinal axis and at least one of the imaging planes is not aligned with the longitudinal axis of the imaging body.

In various embodiments, the endoscopic ultrasonic imaging system may include alignment structure having a stop positioned to align the ultrasonic array of the imaging device with the imaging window of the endoscope; at least one of the imaging planes may be canted with respect to the longitudinal axis of the imaging body; imaging electronics may be connected to the plurality of ultrasonic elements in the ultrasonic array to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array; etc.

In still other embodiments, the imaging electronics may display a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane. The first imaging plane may be perpendicular to the second imaging plane. The imaging electronics may display the first visual image and the second visual image at the same time.

In another aspect, the present invention may provide a method of ultrasonic imaging, the method including advancing an ultrasonic imaging device to an internal body location, wherein the ultrasonic imaging device includes an elongated body defining a longitudinal axis extending from a proximal to a distal end of the body; a first ultrasonic array located proximate the distal end of the body, the first ultrasonic array having a plurality of ultrasonic elements arranged in a two-dimensional array over a selected area of the body, wherein, in a circumferential dimension, the two-dimensional array has two or more adjacent ultrasonic elements arranged over a circumferential segment of the body, and wherein, in a longitudinal dimension, the two-dimensional array has two or more adjacent ultrasonic elements arranged over a longitudinal segment of the body, and wherein the ultrasonic elements of the first ultrasonic array are selectively activatable. The method further includes selectively activating the ultrasonic elements of the ultrasonic array of the imaging device to obtain images in imaging planes, wherein at least one of the imaging planes is aligned with the longitudinal axis and at least one of the imaging planes is not aligned with the longitudinal axis; and displaying the images on a display.

In various embodiments, the method may include one or more of the following: selectively activating the ultrasonic elements may include activating a set of ultrasonic elements in the ultrasonic array to obtain images in an imaging plane that contains the longitudinal axis; selectively activating the ultrasonic elements may include activating a set of ultrasonic elements in the ultrasonic array to obtain images in an imaging plane that is perpendicular to the longitudinal axis; selectively activating the ultrasonic elements may include activating a set of ultrasonic elements in the ultrasonic array to obtain images in an imaging plane that is canted with respect to the longitudinal axis; the ultrasonic imaging device may include a first port channel extending through the body, the first port channel having a first port located between the first ultrasonic array and the distal end of the body or between the first ultrasonic array and the proximal end of the body, and the method may include selectively activating a set of ultrasonic elements in the ultrasonic array to obtain images in an imaging plane that intersects with the first port; advancing the ultrasonic imaging device may include advancing the device through an imaging channel of an endoscope; etc.

In still other embodiments, the methods may include selectively activating two different sets of ultrasonic elements in the ultrasonic array to obtain images in a first imaging plane and a second imaging plane, wherein the first and second imaging planes are not coplanar. The first imaging plane may be perpendicular to the second imaging plane.

In another aspect, the present invention may provide an ultrasonic imaging device that includes an elongated body defining a longitudinal axis extending from a proximal to a distal end of the body; a first ultrasonic array located proximate the distal end of the body, the first ultrasonic array comprising a plurality of ultrasonic elements arranged in a two-dimensional array over a selected area of the body, wherein, in a circumferential dimension, the two-dimensional array includes two or more adjacent ultrasonic elements arranged over a circumferential segment of the body, and wherein, in a longitudinal dimension, the two-dimensional array includes two or more adjacent ultrasonic elements arranged over a longitudinal segment of the body, and wherein the ultrasonic elements of the first ultrasonic array are selectively activatable. The device may include an optional distal port channel extending through the body, the distal port channel having a distal port located between the first ultrasonic array and the distal end of the body; an optional proximal port channel extending through the body, the proximal port channel having a proximal port located between the first ultrasonic array and the proximal end of the body; an optional first side port channel extending through the body, the first side port channel having a first side port that is radially displaced from the first ultrasonic array, the first side port being located between the distal port and the proximal port along the longitudinal axis; and an optional balloon attached to the body, the balloon extending over the first ultrasonic array, wherein the balloon includes a gas permeable membrane.

In various embodiments, the devices of the present invention may include one or more of the following features: a terminal port channel may extend through the body, the terminal port channel having a terminal port located at the distal end of the body; the distal port and the proximal port may intersect with a plane that contains the longitudinal axis; the first ultrasonic array may intersect with the plane that contains the longitudinal axis and intersects with the distal port and the proximal port; the first side port and the first ultrasonic array may intersect with a plane that is perpendicular to the longitudinal axis of the body; a second side port channel may extend through the body, the second side port channel comprising a second side port that is radially displaced from the first ultrasonic array, the second side port being located between the distal port and the proximal port along the longitudinal axis, wherein the first ultrasonic array is located between the first side port and the second side port; the first side port and the second side port (if provided) may intersect with a plane that is perpendicular to the longitudinal axis of the body; and the first ultrasonic array may intersect with the plane that also intersects with the first side port and the second side port.

In still further embodiments, the imaging devices may also include one or more of the following feature: a second ultrasonic array may be located proximate the distal end of the body, wherein the second ultrasonic array includes a plurality of ultrasonic elements arranged in a two-dimensional array over a selected area of the body, wherein, in a circumferential dimension, the two-dimensional array includes two or more adjacent ultrasonic elements arranged over a circumferential segment of the body, and wherein, in a longitudinal dimension, the two-dimensional array includes two or more adjacent ultrasonic elements arranged over a longitudinal segment of the body, and wherein the ultrasonic elements of the second ultrasonic array are selectively activatable; the second ultrasonic array may be located on an opposite side of the body from the first ultrasonic array; a second distal port channel may extend through the body, the second distal port channel including a second distal port located between the second ultrasonic array and the distal end of the body; a second proximal port channel may extend through the body, the second proximal port channel including a second proximal port located between the second ultrasonic array and the proximal end of the body; a second side port channel may extend through the body, the second side port channel including a second first side port that is located between the first ultrasonic array and the second ultrasonic array; a second balloon may be attached to the body, the second balloon extending over the second ultrasonic array, wherein the second balloon includes a gas permeable membrane; the balloon may extend over the second ultrasonic array in addition to the first ultrasonic array; and, wherein, in the circumferential dimension, the two-dimensional array of the first ultrasonic array includes adjacent ultrasonic elements arranged around the entire circumference of the body.

In another aspect, the present invention may provide an ultrasonic imaging system that includes an ultrasonic imaging device of the present invention and imaging electronics connected to the plurality of ultrasonic elements in the first ultrasonic array of the imaging device to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array.

The systems described above may include one or more of the following features: the imaging electronics may display a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane; the first imaging plane may be perpendicular to the second imaging plane; the imaging electronics may display the first visual image and the second visual image at the same time; etc.

In another aspect, the present invention may provide an endoscopic ultrasonic imaging system that includes an endoscope having an endoscope body defining a longitudinal axis extending from a proximal end to a distal end of the endoscope body; an imaging channel extending through the endoscope body, the imaging channel having an imaging window and, optionally, an alignment structure; and an imaging device sized for advancement through the imaging channel. The imaging device may include an imaging body defining a longitudinal axis extending from a proximal end to a distal end of the imaging body; an ultrasonic array located proximate the distal end of the imaging body, the ultrasonic array including a plurality of ultrasonic elements arranged in a two-dimensional array over a selected area of the imaging body, wherein, in a circumferential dimension, the two-dimensional array includes two or more adjacent ultrasonic elements arranged over a circumferential segment of the body, and wherein, in a longitudinal dimension, the two-dimensional array includes two or more adjacent ultrasonic elements arranged over a longitudinal segment of the body, and wherein the ultrasonic elements of the ultrasonic array are selectively activatable. The system may further include an optional balloon extending over the ultrasonic array, wherein the balloon includes a gas permeable membrane.

The endoscopic ultrasonic imaging system described in the preceding paragraph may include one or more of the following features: the alignment structure may include a stop positioned to align the ultrasonic array of the imaging device with the imaging window of the endoscope; the imaging electronics may be connected to the plurality of ultrasonic elements in the ultrasonic array to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array; the imaging electronics (if provided) may display a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane; the first imaging plane may be perpendicular to the second imaging plane; the imaging electronics may display the first visual image and the second visual image at the same time.

In another aspect, the present invention may provide a method of ultrasonic imaging, the method including: advancing an ultrasonic imaging device according to the present invention to an internal body location; operating an ultrasonic array of the imaging device to obtain a visual image; and displaying the visual image on a display.

The method described in the preceding paragraph may include one or more of the following: operating the ultrasonic array may include selectively activating a set of ultrasonic elements in the ultrasonic array to obtain an imaging plane that intersects the ultrasonic array; operating the ultrasonic array may include selectively activating a two different sets of ultrasonic elements in the ultrasonic array to obtain first and second imaging planes, both of which intersect the ultrasonic array, wherein the first and second imaging planes are not coplanar; the first imaging plane may be perpendicular to the second imaging plane; advancing the ultrasonic imaging device may include advancing the device through an imaging channel of an endoscope.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a device with “an” ultrasonic array may include one or more ultrasonic arrays.

The term “and/or” means one or all of the listed elements or a combination of any two or more of the listed elements.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

The present invention will be further described with reference to the views of the drawing, wherein:

FIG. 1 is a longitudinal cross-sectional view of a portion of the ultrasonic imaging device of FIG. 2 taken along the longitudinal axis 11 in FIG. 2.

FIG. 2 is a plan view of one exemplary ultrasonic imaging device according to the present invention.

FIG. 3 is a plan view of some exemplary planar sectional views that may be obtained using an ultrasonic array in an ultrasonic imaging device according to the present invention.

FIG. 4 is an end view of the ultrasonic imaging device of FIG. 3 depicting the exemplary planar sectional views from a different perspective.

FIG. 5 is a partial cross-sectional view of a portion of a different exemplary ultrasonic imaging device including two ultrasonic arrays according to the present invention.

FIG. 6 is a side view of an exemplary endoscope with an ultrasonic imaging device located in an imaging channel provided in the endoscope.

FIG. 7 is a block diagram depicting exemplary components of an exemplary imaging system according to the present invention.

### DETAILED DESCRIPTION OF EXEMPLARY EMBODIMENTS

In the following detailed description of exemplary embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

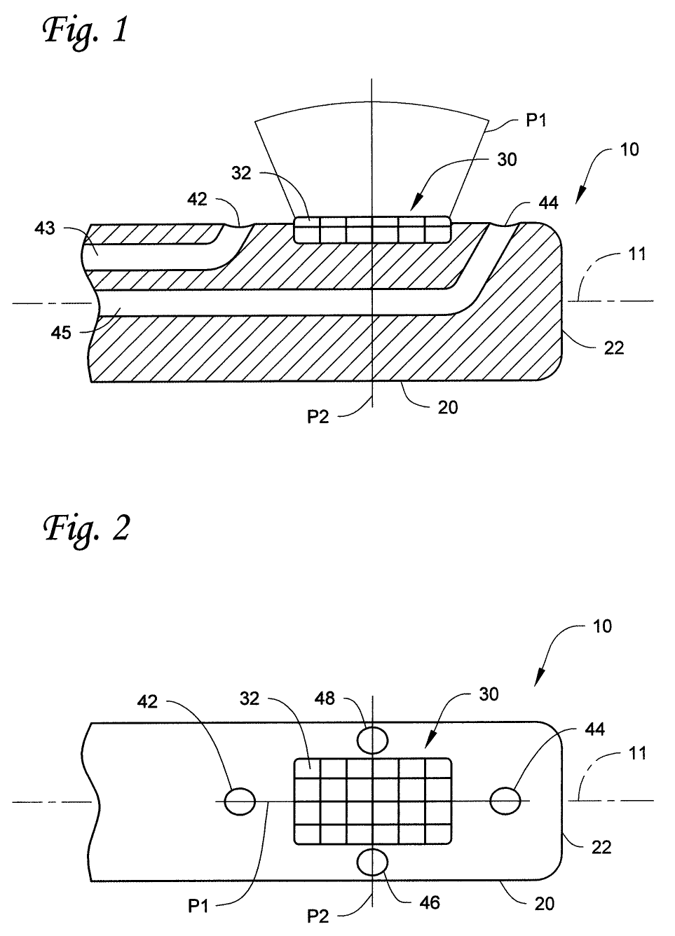

One exemplary embodiment of an ultrasonic imaging device 10 according to the present invention is depicted in connection with FIGS. 1 & 2. In the views of FIGS. 1 & 2 only the distal portion of the device 10 is depicted, although it will be understood that the device 10 includes a proximal end (not shown) that is positioned along the longitudinal axis 11. The device 10 includes a body 20 that may be rigid or flexible and that extends along the longitudinal axis 11 to a distal end 22. Although the body 20 may be circular in a cross-section taken perpendicular to the longitudinal axis 11, other shapes may also be used for the body 20.

The device 10 also includes an ultrasonic array 30 located on a selected area of the body 20. In addition, the device 10 may also include various ports that are located around the ultrasonic array 30. The embodiment depicted in FIGS. 1 & 2 includes a proximal port 42, a distal port 44, a first side port 46, and a second side port 48. Although the ultrasonic array 30 is depicted as curved about the longitudinal axis 11, it may alternatively be flat or take any other suitable shape.

The proximal port 42 may preferably be located at the end of a proximal port channel 43 (see FIG. 1) that extends in the proximal direction away from the distal end 22 of the body 20. The distal port 44 may preferably be located at the end of a distal port channel 45 (see FIG. 1) that extends in the proximal direction away from the distal end 22 of the body 20. The first side port 46 and the second side port 48 may also preferably be located at the ends of channels (not shown) that also extend proximally (i.e., away from the distal end 22 of the body 20). Although the ports are described as being served by individual channels, in some embodiments, two or more ports may share a common channel.

The ultrasonic array 30 may preferably be constructed of a plurality of ultrasonic elements 32 arranged in a two-dimensional array over a selected area of the body 20. In a circumferential dimension, the depicted two-dimensional array 30 includes four adjacent ultrasonic elements 32 arranged over a circumferential segment of the body 20. In a longitudinal dimension, the depicted two-dimensional array 30 includes six adjacent ultrasonic elements 32 arranged over a longitudinal segment of the body 20. It should be understood that the particular number of ultrasonic elements and the number of rows in which they are arranged is exemplary only (for this and all other embodiments described herein) and that any suitable number of ultrasonic elements and arrays with any suitable dimensions may be used in connection with the present invention.

The ultrasonic elements 32 of the array 30 may preferably be selectively activatable. In one embodiment, selective activation of the ultrasonic elements may mean that each individual element 32 is capable of transmitting and/or receiving ultrasonic energy independently of the other ultrasonic elements 32 in the array 30. In another variation, selective activation of the ultrasonic elements 32 may involve activation of selected sets of the ultrasonic elements 32. For example, the set of ultrasonic elements 32 forming one of the four longitudinal rows may be selectively activated and/or the set of ultrasonic elements 32 forming one of the six circumferential rows of ultrasonic elements 32 may be selectively activated.

It may be preferred that at least some of the ports be aligned with each other and located in imaging planes that can be provided using the ultrasonic array 30. For example, As seen in FIGS. 1 & 2, it may be advantageous if the proximal port 42 and the distal port 44 are aligned with each other such that the two ports intersect with an imaging plane P1 that is aligned with the longitudinal axis 11 (with imaging plane P1 being seen in a plan view in FIG. 1 and an edge view in FIG. 2). Such an arrangement may be beneficial in providing images of devices, instruments, etc. delivered through the ports 42 and 44.

Similarly, it may be advantageous if the first side port 46 and the second side port 48 are aligned with each other such that the two ports intersect with an imaging plane P2 that is, in the depicted embodiment, oriented generally perpendicular to the longitudinal axis 11 (with only the edges of imaging plane P2 being seen in both FIGS. 1 & 2).

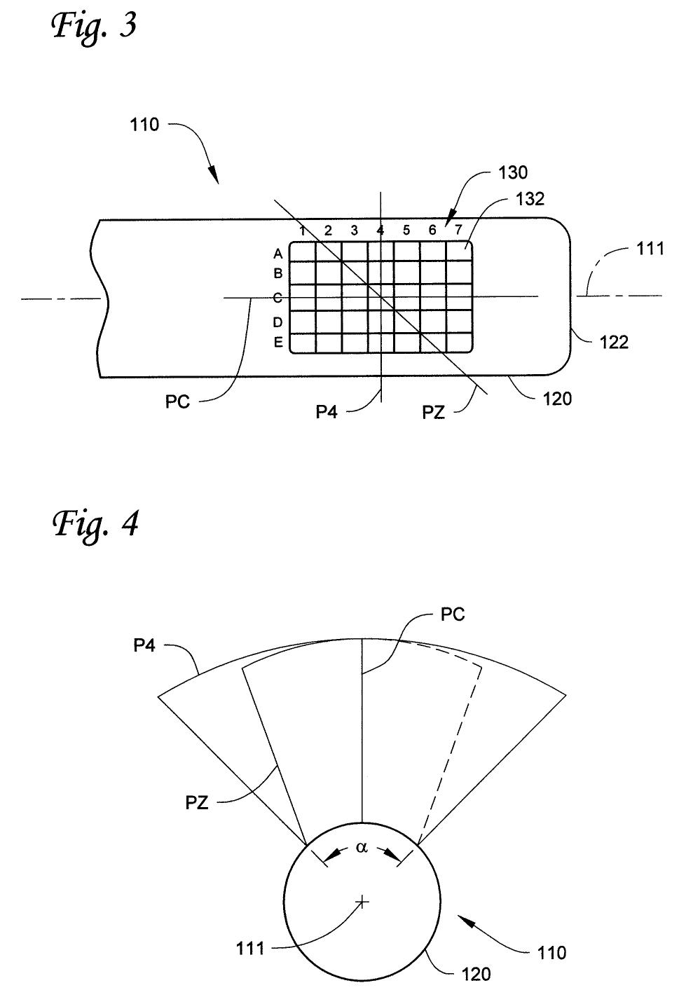

FIGS. 3 & 4 depict an alternative embodiment of an ultrasonic imaging device 110 that includes an ultrasonic array 130. Although the device 110 may include ports, no such ports are depicted to focus on the various imaging planes that may be obtained using the ultrasonic array 130. The depicted array 130 includes ultrasonic elements 132 arranged in five rows (designated by the letters A, B, C, D, E) that extend along the longitudinal dimension (aligned with the longitudinal axis 111). Each longitudinal row of ultrasonic elements 132 includes seven elements 132 such that seven circumferential rows (designated by the numbers 1-7 in FIGS. 3 & 4) are provided.

FIG. 3 is a side view of the device 110 while FIG. 4 is taken along the longitudinal axis 111 (i.e., it is an end view of the distal end 122 of the body 120 of the device 130). If, for example, the longitudinal row C of ultrasonic elements 132 is selectively activated, then an imaging plane PC may be obtained. Imaging plane PC may preferably be parallel to the longitudinal axis 111 and, in some instances, the longitudinal axis 111 may be located with imaging plane PC (where the imaging plane PC extends radially away from the axis 111). Because of its orientation, only the edges of imaging plane PC are seen in both FIGS. 3 & 4.

Alternatively, if the circumferential row 4 of ultrasonic elements 132 is selectively activated, then an image in imaging plane P4 may be obtained. Imaging plane P4 may preferably be perpendicular to the longitudinal axis 111 as depicted in FIGS. 3 & 4. Because of its orientation, imaging plane P4 is seen in FIG. 4, but only an edge of the plane P4 is seen in FIG. 3. Because the circumferential segment over which the ultrasonic array 130 extends is only a portion of the circumference of the body 120, the image provided by the ultrasonic elements 132 of circumferential row 4 in imaging plane P4 is limited to a radial segment α (alpha) of the plane P4. If the array 130 extended further around the body 120, then the radial segment would be larger.

If the selective activation scheme used in connection with the ultrasonic array 130 is capable of activating elements 132 that are in different longitudinal and circumferential rows, then it may be possible to obtain images in imaging planes that are not aligned with or perpendicular to the longitudinal axis 111. Such imaging planes may be referred to as canted imaging planes. One example of a canted imaging plane PZ is depicted in FIGS. 3 & 4, where the ultrasonic elements in different longitudinal and circumferential rows are selectively activated to provide an image in an imaging plane PZ, an edge of which is seen in FIG. 3 and which is seen as canted relative to the longitudinal axis 111 in FIG. 4 (with the portion of imaging plane PZ located behind imaging plane P4 depicted in broken lines).

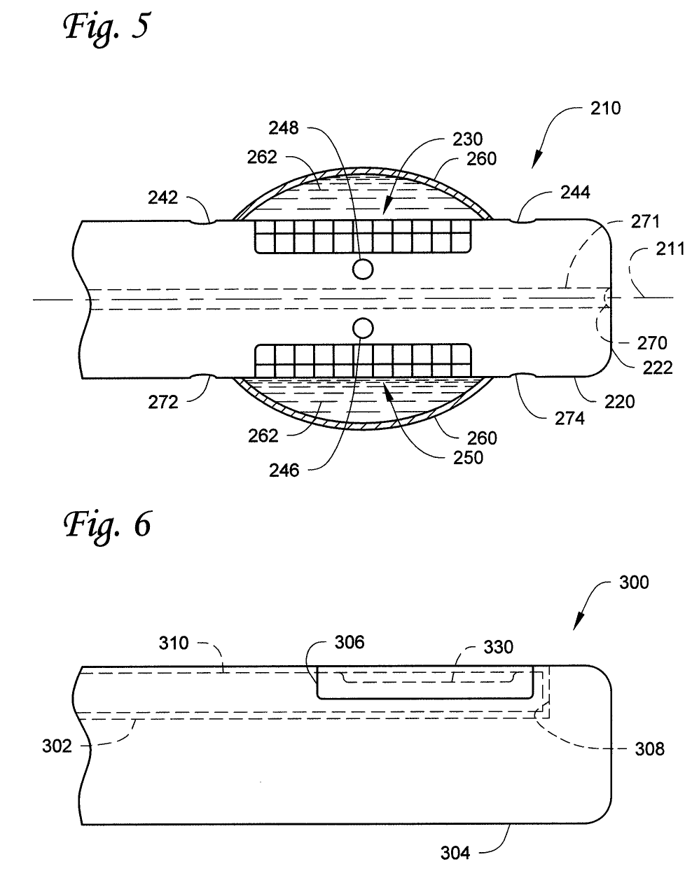

Another alternative embodiment of an ultrasonic imaging device 210 is depicted in FIG. 5 and includes a first ultrasonic array 230 on one side of the body 220 and a second ultrasonic array 250 on an opposite side of the device 210. Although the two ultrasonic arrays 230 & 250 are depicted as being located directly opposite each other, they may be longitudinally offset from each other along the longitudinal axis 211.

Another optional feature depicted in connection with the embodiment of FIG. 5 is a terminal port 270 and associated terminal port channel 271 (depicted in broken lines in FIG. 5), where the terminal port 270 is located at the distal end 222 of the body 220 and the terminal port channel 271 extends away from the distal end 222 and towards the proximal end (not shown) of the device 210. Although only one terminal port 270 is depicted, it should be understood that the devices of the present invention may be provided with more than one terminal port.

In addition to the terminal port 270, the device 210 also includes optional proximal ports 242 and 272 located on the proximal side of the ultrasonic arrays 230 & 250 (i.e., between the ultrasonic arrays 230 & 250 and the proximal end (not shown) of the body 220). The depicted device 210 also includes optional distal ports 244 and 274 located distally of the ultrasonic arrays 230 & 250 (i.e., between the ultrasonic arrays 230 & 250 and the distal end 222 of the body 220). Optional side ports 248 are also provided in the device 210. Although the channels associated with these different ports are not shown, the ports are preferably located at the ends of channels that extend in the proximal direction such that instruments, materials, etc. can be delivered to the ports as described elsewhere herein.

Yet another optional feature depicted in connection with the embodiment of FIG. 5 is the balloon 260 (shown in cross-section only) located over both of the ultrasonic arrays 230 & 250. The balloon 260 may preferably be filled with a liquid 262 (e.g., water, saline, etc.) to assist with the transmission of ultrasonic energy to and from the arrays 230 and 250.

As discussed herein, one potential problem associated with liquid-filled balloons is the presence of gas bubbles in the liquid. The balloon 260 is, however, preferably formed with a gas-permeable membrane that allows the passage of gas out of the liquid in the balloon 260, but prevents the passage of liquids. Examples of some potentially suitable gas-permeable membranes may be described in, e.g., U.S. Pat. No. 5,251,619 (Lee) and U.S. Pat. No. 6,334,064 (Fiddian-Green) as well as U.S. Patent Application Publication No. US 2005/0049331 (Coutinho et al.). The entire balloon 260 may be constructed of the gas permeable membrane or only a portion of the balloon 260 may be constructed of a gas permeable membrane.

Although only one balloon 260 is depicted in FIG. 5, it should be understood that each of the ultrasonic arrays 230 & 250 may be provided with a different balloon. Also, the balloon 260 may be attached to and sealed by any suitable technique (e.g., O-rings, bands, etc.), one example of which is described in U.S. Pat. No. 6,338,717 (Ouchi). Where the arrays 230 & 250 are provided with separate balloons, it may be possible to provide ports (and associated channels) that are located between the arrays 230 & 250).

FIG. 6 depicts an exemplary embodiment of an endoscope 300 that may not, itself, include ultrasonic imaging apparatus. The endoscope 300 does, however, include an imaging channel 302 (depicted in broken lines) formed in the body 304. The imaging channel 302 preferably extends to a window 306 formed in the endoscope 300 that allows ultrasonic energy to pass into and out of the imaging channel 302. An ultrasonic imaging device 310 may be advanced through the imaging channel 302 such that one or more ultrasonic arrays 330 may be positioned to emit and receive ultrasonic energy through the window 306 to provide ultrasonically-generated images to a user. The endoscope 300 may include alignment structure 308 (depicted in broken lines), such as a terminal end of the imaging channel 302 that may assist in properly locating the imaging device 310 relative to the window 306.

A potential benefit of interchangeable imaging devices 310 and endoscopes 300 in which they can be used may be found in the ability to tailor the imaging to the particular procedure and/or preference of the practitioner. The various imaging devices 310 that could be advanced down the imaging channel 302 could have different imaging properties (e.g., number of ultrasonic elements, imaging frequencies, etc.) to give different views (e.g., tissue depth of image, resolution, etc.).

Those different views may be advantageous. For example, at one point during a procedure, a practitioner may prefer an image that provides increased tissue depth with less resolution. At another point during the procedure, the practitioner might prefer an image with increased resolution, but less tissue depth. Use of a system such as that depicted in FIG. 6 may be advantageous because, rather than exchanging one endoscope for a different endoscope, the practitioner may need to merely exchange one imaging device 310 in the imaging channel 302 for a different imaging device 310. In contrast, exchanging entire endoscopes to obtain different images will typically require that the practitioner advance the second endoscope to the same position as the first endoscope (which may be difficult to accomplish).

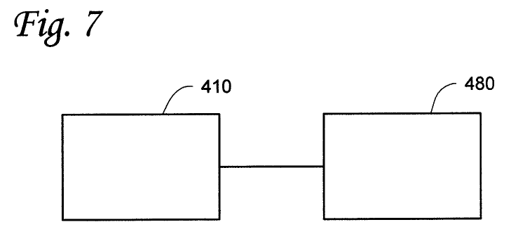

FIG. 7 is a block diagram of one exemplary ultrasonic imaging system of the present invention including an ultrasonic imaging device 410 and imaging electronics 480 that is preferably connected to the ultrasonic elements in the ultrasonic array or arrays provided on or in the ultrasonic imaging device 410. The imaging electronics 480 is preferably capable of transmitting electrical signals to a first transmitter set of ultrasonic elements in the imaging device 410 and receive electronic signals from a first receiver set of ultrasonic elements in the imaging device 410. The imaging electronics 480 may preferably display a first visual image based on the electronic signals received from the first receiver set of ultrasonic elements of the ultrasonic imaging device 410. The first visual image may preferably be located in a first imaging plane that intersects the ultrasonic array containing the transmitter/receiver elements used to generate the image.

In some embodiments, the imaging electronics 480 may display a second visual image based on electronic signals received from a second receiver set of ultrasonic elements of the imaging device 410. The second visual image may preferably be located in a second imaging plane that intersects the ultrasonic array containing the transmitter/receiver elements used to generate the second image. The second imaging plane may preferably not be coplanar with the first imaging plane. In some embodiments, the second imaging plane may preferably be perpendicular to the first imaging plane.

The imaging electronics 480 may include one or more display devices capable of providing a visual image to a user where a visual image is an image that can be detected by the eyes of a human user. The display devices may be provided in any suitable form (e.g., LCD displays, CRT monitors, plasma displays, heads-up displays, etc.).

The imaging electronics 410 may be provided in any suitable form and may, for example, preferably include memory and a controller. The controller may be, e.g., an Application Specific Integrated Circuit (ASIC) state machine, a gate array, and/or may include a microprocessor. The imaging electronics 480 are preferably configured to operate the ultrasonic array of imaging device 410 as well as any display devices.

The display device or devices may preferably be capable of providing two or more different visual images at the same time. Referring to FIGS. 3 & 4, for example, the display devices may preferably be capable of providing images taken in two or more different imaging planes (e.g., imaging planes PC, P7, and PZ) at the same time.

In some instances, the ultrasonic imaging devices and systems of the present invention may be used to obtain three-dimensional (3D) images of the tissue being imaged. Using conventional ultrasonic imaging devices, such as those described in U.S. Pat. No. 6,171,248 (Hossack et al.), requires rotation and/or translation of the actual imaging device to provide the images required to construct a 3D image. In contrast, devices with ultrasonic arrays such as those described in connection with the present invention may be capable of obtaining the image data needed to construct 3D images without requiring movement of the device itself.

Referring back to, e.g., FIGS. 3 & 4, the ultrasonic elements in the arrays on the devices of the present invention may be selectively activated to obtain a series of images oriented longitudinally, radially, etc. For example, The ultrasonic elements 132 in the different rows A, B, C, D, E of array 130 in FIG. 3 may be used to obtain a series of images in image planes extending radially from the longitudinal axis 111 (with each row of ultrasonic elements 132 providing a different image). Furthermore, the circumferential rows 1-7 may be used to obtain a series of images in image planes oriented perpendicular to the longitudinal axis 111. These different images may preferably be obtained without requiring movement of the device 110 and the different images may be used to construct a 3D image of the tissue proximate the device 110.

Although not expressly described herein, the construction and operation of ultrasonic transducers and the imaging systems that use them are known to those skilled in the art and will not be explicitly described herein. For example, U.S. Pat. No. 6,171,248 (Hossack et al.) and U.S. Pat. No. 6,338,717 (Ouchi) and the references identified therein describe the construction and operation of ultrasonic transducers.

The complete disclosure of the patents, patent documents, and publications cited in the Background, the Detailed Description of Exemplary Embodiments, and elsewhere herein are incorporated by reference in their entirety as if each were individually incorporated.

Exemplary embodiments of this invention have been discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An ultrasonic imaging device comprising:
an elongated body defining a longitudinal axis extending from a proximal to a distal end of the body;
a first ultrasonic array located proximate the distal end of the body, the first ultrasonic array comprising a plurality of ultrasonic elements arranged in a two-dimensional array over a selected area of the body, wherein, in a circumferential dimension, the two-dimensional array comprises two or more adjacent ultrasonic elements arranged over a circumferential segment of the body, and wherein, in a longitudinal dimension, the two-dimensional array comprises two or more adjacent ultrasonic elements arranged over a longitudinal segment of the body, and wherein the ultrasonic elements of the first ultrasonic array are selectively activatable to obtain images in imaging planes, wherein at least one of the imaging planes is aligned with the longitudinal axis and at least one of the imaging planes is not aligned with the longitudinal axis; and
a first port channel extending through the body, the first port channel comprising a first port located between the first ultrasonic array and the distal end of the body or between the first ultrasonic array and the proximal end of the body, wherein at least one of the imaging planes intersects with the first port.

2. A device according to claim 1, the device further comprising a second port channel extending through the body, the second port channel comprising a second port, wherein the first ultrasonic array is located between the first port and the second port, wherein at least one of the imaging planes intersects with the first port, the second port, and the longitudinal axis.

3. A device according to claim 1, the device further comprising a first side port channel extending through the body, the first side port channel comprising a first side port that is radially displaced from the first ultrasonic array, the first side port being located between the distal port and the proximal port along the longitudinal axis, wherein at least one of the imaging planes intersects with the first side port

4. A device according to claim 3, the device further comprising a second side port channel extending through the body, the second side port channel comprising a second side port that is radially displaced from the first ultrasonic array, the second side port being located between the distal port and the proximal port along the longitudinal axis, wherein the first ultrasonic array is located between the first side port and the second side port.

5. A device according to claim 4, wherein the first side port and the second side port intersect with at least one of the imaging planes.

6. A device according to claim 1, the device further comprising a terminal port channel extending through the body, the terminal port channel comprising a terminal port located at the distal end of the body, wherein at least one of the imaging planes intersects with the terminal port.

7. A device according to claim 1, wherein, in the circumferential dimension, the two-dimensional array of the first ultrasonic array comprises adjacent ultrasonic elements arranged around the entire circumference of the body.

8. A device according to claim 1, the device further comprising a balloon attached to the body, the balloon extending over the first ultrasonic array, wherein the balloon comprises a gas permeable membrane, wherein gas contained within a liquid located between the body and the gas permeable membrane diffuses out of the liquid through the gas permeable membrane.

9. A device according to claim 1, further comprising a second ultrasonic array located proximate the distal end of the body, wherein the second ultrasonic array comprises a plurality of ultrasonic elements arranged in a two-dimensional array over a selected area of the body, wherein, in a circumferential dimension, the two-dimensional array comprises two or more adjacent ultrasonic elements arranged over a circumferential segment of the body, and wherein, in a longitudinal dimension, the two-dimensional array comprises two or more adjacent ultrasonic elements arranged over a longitudinal segment of the body, and wherein the ultrasonic elements of the second ultrasonic array are selectively activatable to obtain images in imaging planes, wherein at least one of the imaging planes is aligned with the longitudinal axis and at least one of the imaging planes is not aligned with the longitudinal axis.

10. A device according to claim 9, wherein the second ultrasonic array is located on an opposite side of the body from the first ultrasonic array.

11. A device according to claim 9, further comprising a third port channel extending through the body, the third port channel comprising a third port, wherein at least one of the imaging planes obtained using the second ultrasonic array intersects with the third port.

12. A device according to claim 9, the device further comprising a fourth port channel extending through the body, the fourth port channel comprising a fourth port, wherein the second ultrasonic imaging array is located between the third port and the fourth port, and wherein at least one of the imaging planes obtained using the second ultrasonic array intersects with the third port, the fourth port, and the longitudinal axis.

13. An ultrasonic imaging system comprising:
an ultrasonic imaging device that comprises:
an elongated body defining a longitudinal axis extending from a proximal to a distal end of the body;
a first ultrasonic array located proximate the distal end of the body, the first ultrasonic array comprising a plurality of ultrasonic elements arranged in a two-dimensional array over a selected area of the body, wherein, in a circumferential dimension, the two-dimensional array comprises two or more adjacent ultrasonic elements arranged over a circumferential segment of the body, and wherein, in a longitudinal dimension, the two-dimensional array comprises two or more adjacent ultrasonic elements arranged over a longitudinal segment of the body, and wherein the ultrasonic elements of the first ultrasonic array are selectively activatable to obtain images in imaging planes, wherein at least one of the imaging planes is aligned with the longitudinal axis and at least one of the imaging planes is not aligned with the longitudinal axis; and
a first port channel extending through the body, the first port channel comprising a first port located between the first ultrasonic array and the distal end of the body or between the first ultrasonic array and the proximal end of the body, wherein at least one of the imaging planes intersects with the first port;
imaging electronics connected to the plurality of ultrasonic elements in the first ultrasonic array to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array.

14. A system according to claim 13, wherein the imaging electronics displays a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane.

15. A system according to claim 14, wherein the first imaging plane is perpendicular to the second imaging plane.

16. A system according to claim 14, wherein the imaging electronics displays the first visual image and the second visual image at the same time.

17. A system according to claim 13, wherein at least one of the imaging planes is canted with respect to the longitudinal axis of the imaging body.

18. An endoscopic ultrasonic imaging system comprising:
an endoscope comprising an endoscope body defining a longitudinal axis extending from a proximal end to a distal end of the endoscope body;
an imaging channel extending through the endoscope body, the imaging channel comprising an imaging window and, optionally, an alignment structure;
an imaging device sized for advancement through the imaging channel, the imaging device comprising:
an imaging body defining a longitudinal axis extending from a proximal end to a distal end of the imaging body;
an ultrasonic array located proximate the distal end of the imaging body, the ultrasonic array comprising a plurality of ultrasonic elements arranged in a two-dimensional array over a selected area of the imaging body, wherein, in a circumferential dimension, the two-dimensional array comprises two or more adjacent ultrasonic elements arranged over a circumferential segment of the body, and wherein, in a longitudinal dimension, the two-dimensional array comprises two or more adjacent ultrasonic elements arranged over a longitudinal segment of the body, and wherein the ultrasonic elements of the ultrasonic array are selectively activatable to obtain images in imaging planes, wherein at least one of the imaging planes is aligned with the longitudinal axis and at least one of the imaging planes is not aligned with the longitudinal axis of the imaging body.

19. A system according to claim 18, wherein the alignment structure comprises a stop positioned to align the ultrasonic array of the imaging device with the imaging window of the endoscope.

20. A system according to claim 18, wherein at least one of the imaging planes is canted with respect to the longitudinal axis of the imaging body.

21. A system according to claim 18, the system further comprising imaging electronics connected to the plurality of ultrasonic elements in the ultrasonic array to transmit electronic signals to a first transmitter set of ultrasonic elements of the plurality of ultrasonic elements and receive electronic signals from a first receiver set of ultrasonic elements of the plurality of ultrasonic elements, wherein the imaging electronics displays a first visual image based on the electronic signals received from the first receiver set of ultrasonic elements of the plurality of ultrasonic elements, wherein the first visual image is located within a first imaging plane that intersects with the first ultrasonic array.

22. A system according to claim 21, wherein the imaging electronics displays a second visual image based on electronic signals received from a second receiver set of ultrasonic elements of the plurality of ultrasonic elements, wherein the second visual image is located within a second imaging plane that intersects with the first ultrasonic array, wherein the second imaging plane is not coplanar with the first imaging plane.

23. A system according to claim 22, wherein the first imaging plane is perpendicular to the second imaging plane.

24. A system according to claim 22, wherein the imaging electronics displays the first visual image and the second visual image at the same time.

25. A method of ultrasonic imaging, the method comprising:
advancing an ultrasonic imaging device to an internal body location, wherein the ultrasonic imaging device comprises:
an elongated body defining a longitudinal axis extending from a proximal to a distal end of the body;
a first ultrasonic array located proximate the distal end of the body, the first ultrasonic array comprising a plurality of ultrasonic elements arranged in a two-dimensional array over a selected area of the body, wherein, in a circumferential dimension, the two-dimensional array comprises two or more adjacent ultrasonic elements arranged over a circumferential segment of the body, and wherein, in a longitudinal dimension, the two-dimensional array comprises two or more adjacent ultrasonic elements arranged over a longitudinal segment of the body, and wherein the ultrasonic elements of the first ultrasonic array are selectively activatable;
selectively activating the ultrasonic elements of the ultrasonic array of the imaging device to obtain images in imaging planes, wherein at least one of the imaging planes is aligned with the longitudinal axis and at least one of the imaging planes is not aligned with the longitudinal axis; and
displaying the images on a display.

26. A method according to claim 25, wherein selectively activating the ultrasonic elements comprises activating a set of ultrasonic elements in the ultrasonic array to obtain images in an imaging plane that contains the longitudinal axis.

27. A method according to claim 25, wherein selectively activating the ultrasonic elements comprises activating a set of ultrasonic elements in the ultrasonic array to obtain images in an imaging plane that is perpendicular to the longitudinal axis.

28. A method according to claim 25, wherein selectively activating the ultrasonic elements comprises activating a set of ultrasonic elements in the ultrasonic array to obtain images in an imaging plane that is canted with respect to the longitudinal axis.

29. A method according to claim 25, wherein selectively activating the ultrasonic elements comprises activating two different sets of ultrasonic elements in the ultrasonic array to obtain images in a first imaging plane and a second imaging plane, wherein the first and second imaging planes are not coplanar.

30. A method according to claim 29, wherein the first imaging plane is perpendicular to the second imaging plane.

31. A method according to claim 25, wherein the ultrasonic imaging device comprises a first port channel extending through the body, the first port channel comprising a first port located between the first ultrasonic array and the distal end of the body or between the first ultrasonic array and the proximal end of the body, the method further comprising selectively activating a set of ultrasonic elements in the ultrasonic array to obtain images in an imaging plane that intersects with the first port.

32. A method according to claim 25, wherein advancing the ultrasonic imaging device comprises advancing the device through an imaging channel of an endoscope.
